(19) United States
(12) Patent Application Publication
(10) Pub. No.: US 2012/0052138 A1
(43) Pub. Date: Mar. 1, 2012

(54) COMPOSITION COMPRISING GREEN TEA EXTRACT

(76) Inventors: Pil Joon Park, Gyeonggi-do (KR); Chae Wook Kim, Gyeonggi-do (KR); Eui Seok Shin, Gyeonggi-do (KR); Si Young Cho, Seoul (KR); Lang Gook Yoo, Gyeonggi-do (KR); Yu Jin Oh, Gyeonggi-do (KR); Dae Bang Seo, Gyeonggi-do (KR); Chan Su Rha, Gyeonggi-do (KR); Jin Oh Chung, Gyeonggi-do (KR); Sang Jun Lee, Gyeonggi-do (KR)

(21) Appl. No.: 13/319,494

(22) PCT Filed: May 19, 2010
(86) PCT No.: PCT/KR2010/003162

§ 371 Date: Nov. 8, 2011

(30) Foreign Application Priority Data
May 19, 2009 (KR) 10-2009-0043571

(51) Int. Cl. A61K 36/82; A61P 9/00; A61P 3/06; A61P 9/10; A61P 3/04; A61P 3/10

(52) U.S. Cl. 424/729

(57) ABSTRACT

The present invention relates to a composition containing a green tea extract as an active ingredient. The composition is effective in treating or preventing obesity and the like and can be beneficially employed in various ways in fields such as foodstuffs and medicine.

### TECHNICAL FIELD

The present disclosure relates to a composition comprising a green tea extract as an active ingredient.

### BACKGROUND ART

With the change in lifestyles as well as westernized eating habits, and adult diseases caused by accumulation of lipids in the body, such as obesity, hyperlipidemia, hypertension and arteriosclerosis, are becoming serious problems of the modern people. Obesity is mainly caused by the intake of high-fat, high-protein diet. Although overweight requires controlled diet, it is not easy to practice in daily lives. Frequently, an initially successful body weight loss is reversed, for example, by the yo-yo effect. Accordingly, for maintenance or reduction of body weight, consistent effort and care are required over the long term.

For body weight control, an anti-obesity therapy of reducing by weight through adequate medication and healthy exercise is required. The anti-obesity therapy refers to a diet therapy or controlled diet performed by obese people or those who want to stay in shape by reducing body weight. Occasionally, people tend to resort surgical means such as liposuction or medication in order to achieve the body weight loss in short period of time, and side effect occurs often therefrom.

Since most obese patients receive treatment after the onset of the cause, complications are often accompanied. Furthermore, since the therapeutic effect can vary depending on age and sex and from person to person, development of an anti-obesity agent ensuring prevention and treatment of obesity is keenly needed for the modern people.

### DISCLOSURE

### Technical Problem

An embodiment of the present disclosure is directed to providing a composition containing a first flush green tea extract.

Another embodiment of the present disclosure is directed to providing a composition containing a green tea extract with a total catechin content of 20-40 wt %.

### Technical Solution

A composition according to an embodiment of the present disclosure contains a first flush green tea extract as an active ingredient. In an embodiment, the composition according to the present disclosure contains a green tea extract with a total catechin content of 20-40 wt % as an active ingredient.

### Advantageous Effects

The composition according to the present disclosure comprising a green tea extract as an active ingredient is effective for treatment or prevention of, for example, obesity.

### DESCRIPTION OF DRAWINGS

FIG. 1 is a flow diagram illustrating the procedures of tea catechin extraction, hot water extraction and alcohol extraction.

FIG. 2 shows a result of measuring the fat degrading ability of a green tea extract at various concentrations in adipocytes.

FIG. 3 shows the change in body weight depending on administration of a green tea extract.

FIG. 4 shows the final body weight depending on administration of a green tea extract.

FIG. 5 shows the epididymal fat weight per average body weight depending on administration of a green tea extract.

FIG. 6 shows the result of an organ and tissue toxicity test of a green tea extract.

### BEST MODE

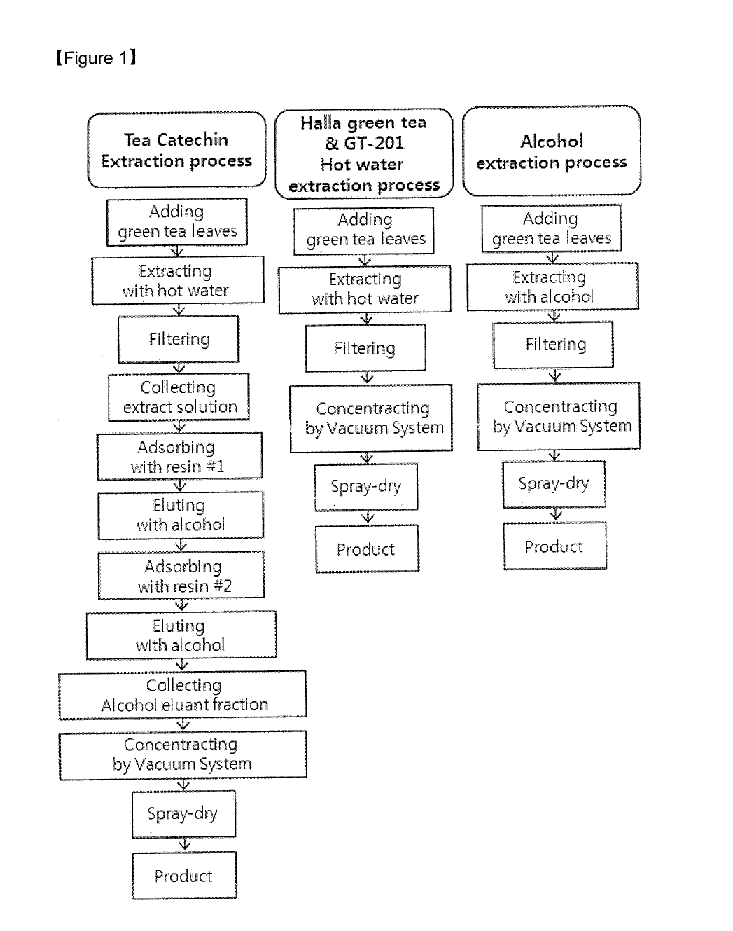

The present disclosure provides a composition comprising a green tea extract as an active ingredient. The method for extracting the green tea extract is not particularly limited. In an exemplary embodiment, the extraction may be carried out using hot water or a C₁-C₅ lower alcohol extract. For example, the green tea extract may be a hot water extract or an ethanol extract. Specifically, it may be a hot water extract of first flush green tea. The hot water extract of first flush green tea may be obtained, for example, through the procedure shown in FIG. 1. Specifically, the product may be obtained following addition of green tea leaves, hot water extraction, filtration, vacuum concentration and spray drying.

In an exemplary embodiment, the composition according to the present disclosure may comprise a first flush green tea extract as an active ingredient. The first flush green tea is sweet and less bitter since the first flush green tea has a higher content of an amino acid than the ordinary green tea when extracted according to the standard procedure. The amino acid is theanine. The content of theanine, which gives the “savory” taste, is identified to be about 2 times higher in the first flush green tea than normal green tea without artificially increasing the theanine content. Also, the extract of the first flush green tea has a considerably higher content of the important catechin, epigallocatechin gallate (EGCG), than the ordinary green tea extract.

The first flush green tea extract according to the present disclosure contains the ingredients highly related with obesity, such as catechin, caffeine and theanine, in high contents. Since these ingredients exist as they are, that is without artificial mixing, they do not interfere with each other but provide superior effect in treating and preventing obesity as well as excellent flavor.

As used herein, the “first flush green tea”, also called first flush tea, spring tea or first tea, refers to the green tea harvested for the first time of the year. In Korea, the first flush green tea is usually harvested between April and May. In general, the first flush green tea is harvested manually in order to preserve its inherent characteristics as much as possible. Thus, the yield is very low and its price is very high. In the present disclosure, the term “ordinary green tea” is used to distinguish from the first flush green tea. The ordinary green tea refers to the green tea harvested after the first flush green tea, from May to autumn.

The present disclosure also provides a composition comprising a green tea extract with a total catechin content of 20-40 wt %, specifically 25-35 wt %, based on the total content of the extract as an active ingredient. The catechin includes epigallocatechin (EGC), epicatechin (EC), epigallocatechin gallate (EGCG), epicatechin gallate (ECG), etc. In an exemplary embodiment, the green tea extract satisfying the above total catechin content may be a first flush green tea extract described above.

In relation to obesity among various adult diseases, accumulation of the highly toxic, oxidized low-density lipoprotein (oxid-LDL) in the intima of blood vessels in the form of cholesterol or cholesteryl ester leads to the formation of foam cells, causing arteriosclerosis. The tea catechins strongly inhibit the oxidation of low-density lipoprotein (LDL). Accordingly, the composition comprising a green tea extract or a first flush green tea extract according to the present disclosure may be a composition for treatment or prevention of arteriosclerosis.

Also, catechins are known to have an anti-obesity activity by reducing serum and liver cholesterol level, suppressing cholesterol reuptake and inhibiting histamine release by mast cells. Accordingly, the composition comprising a green tea extract or a first flush green tea extract according to the present disclosure may be an anti-obesity composition.

In addition, catechin suppresses body fat accumulation by interrupting the action of digestive enzymes in the small intestine and thus inhibiting the absorption of and helping the excretion of surplus nutrients. It is because the catechins reduce blood insulin level, thus lowering blood sugar and reducing body fat. Accordingly, the composition comprising a green tea extract or a first flush green tea extract according to the present disclosure may be a composition for treatment or prevention of diabetes, hyperlipidemia or hypertension.

Another advantage of catechin is its excellent detoxifying activity of inactivating the toxicity and detriment caused by drug abuse. In addition, it is known to have no side effect even when ingested over a long period of time in the form of, e.g., tea. This pharmacological activity arises from the abundant hydroxyl (—OH) groups of catechin, which allow modification and inhibition of other substances through easily binding therewith.

The green tea extract used in the present disclosure has a relatively higher total catechin content as compared to the existing ordinary green tea extract. In particular, it contains EGCG, which is the most important catechin, up to 2 times when compared to the ordinary green tea extract. In an exemplary embodiment, the green tea extract according to the present disclosure has an EGCG content of 7-20 wt %, specifically 10-15 wt %, based on the total weight of the extract.

In an exemplary embodiment, the green tea extract according to the present disclosure has a caffeine content of 2.5-4.5 wt % based on the total weight of the extract. That is to say, the content of caffeine which exhibits excellent fat degrading activity is about 1.5 times higher than the existing ordinary green tea extract.

Among the methylxanthine derivatives, which are alkaloids found in green tea, caffeine, which is also used as vasodilator or psychoactive drug, stimulates the central nervous system, being selectively controlled by catechin and theanine, although the effect does not last long. As such, caffeine exhibits various pharmacological activities, including stimulation, cardiac tonification, diuresis, alleviation of fever, astringency, etc. Especially, by inhibiting increase of cholesterol in the human body together with polyphenols, it treats or prevents angina, myocardial infarction, etc. Accordingly, the composition comprising a green tea extract or a first flush green tea extract according to the present disclosure may be a composition for treatment or prevention of angina or myocardial infarction.

In an exemplary embodiment, the green tea extract according to the present disclosure may further comprise 4.5-10 wt % of amino acids based on the total weight of the extract. Especially, theanine, accounting for more than half of green tea amino acids, is hardly found in other plants. Theanine is an important component that determines the taste and efficacy of green tea and is reported to have various physiological activities. For example, theanine is being spotlighted in various fields as it is known to control the stimulation by caffeine, relieve tension and stress and enhance immunity. In an exemplary embodiment, the green tea extract according to the present disclosure comprises 2-5 wt %, specifically 2.5-3.5 wt %, of theanine based on the total weight of the composition.

In the following examples, experiment was carried out using the hot water extract of first flush green tea harvested in Jeju Island between April and May. The first flush green tea hot water extract was treated to cultured adipocytes at various concentrations and fat degrading effect was investigated by measuring the increase in glycerol and free fatty acid. As a result, the first flush green tea hot water extract showed excellent fat degrading effect as compared to tea catechin (70%) used as control.

Also, diet experiment was carried out using mouse. Specifically, the experiment was carried out for 8 weeks after dividing mice into normal diet group, high-fat diet group, high-fat diet+tea catechin group, high-fat diet+first flush green tea hot water extract (½ of tea catechin) group, high-fat diet+first flush green tea hot water extract (same quantity as tea catechin) group, and high-fat diet+first flush green tea hot water extract (2 times the volume of tea catechin) group. As a result, the high-fat diet+tea catechin group showed no special body weight loss effect, whereas the groups to which the high-fat diet and the first flush green tea hot water extract (same quantity or 2 times that of tea catechin) showed significant body weight decrease. Accordingly, it can be seen that the first flush green tea extract according to the present disclosure is effective for body weight loss.

In an exemplary embodiment, the present disclosure provides a food additive or a functional food comprising the green tea extract according to the present disclosure.

The food additive or functional food comprising the green tea extract may be in various forms. For example, it may be processed into fermented milk, cheese, yogurt, juice, probiotic products, dietary supplements, etc., as well as various food additives.

In an exemplary embodiment, the green tea extract may comprise other ingredients that may enhance its major effect desired by the present disclosure within the range not negatively affecting it. For example, such additives as fragrance, pigment, sterilizer, antioxidant, antiseptic, humectant, thickener, mineral, emulsifier, synthetic polymer, etc. may be further included to improve physical properties. In addition, auxiliary ingredients such as water-soluble vitamin, oil-soluble vitamin, polypeptide, polysaccharide, seaweed extract, etc. may be further included. Those skilled in the art will select those ingredients without special difficulty considering the desired formulation or purpose, and their addition amount can be selected within the range not negatively affecting the purpose and effect of the present disclosure. For instance, the above ingredients may be added in an amount of 0.01-5 wt %, more specifically 0.01-3 wt % based on the total weight of the composition.

The extract according to the present disclosure may be prepared into various formulations, including solution, emulsion, viscous mixture, tablet, powder, etc., and may be administered by various methods, including simple drinking, injection, spraying, squeezing, etc.

The present disclosure also provides a pharmaceutical composition comprising the green tea extract. The pharmaceutical composition comprising the extract according to the present disclosure has body weight control, blood sugar lowering and blood cholesterol lowering effects.

When the extract according to the present disclosure is used for medical use, a commonly used organic or inorganic carrier may be added to the extract as an active ingredient to form a solid, semisolid or liquid formulation for oral or parenteral administration.

Examples of the formulation for oral administration include tablet, pill, granule, soft/hard capsule, powder, fine granule, dust, emulsion, syrup, pellet, etc. And, examples of the formulation for parenteral administration include injection, drip, ointment, lotion, spray, suspending agent, emulsion, suppository, etc. The formulation of the active ingredient may be achieved easily according to the commonly employed method, and other commonly used adjuvants such as surfactant, vehicle, colorant, fragrance, preservative, stabilizer, buffer and suspending agent maybe used appropriately.

The pharmaceutical composition according to the present disclosure may be administered orally or parenterally, e.g. rectally, topically, transdermally, intravenously, intramuscularly, intraabdominally or subcutaneously.

The administration dosage of the active ingredient may vary depending on the age, sex and body weight of the recipient, particular disease or pathological condition to be treated, severity of the disease or pathological condition, route of administration, and discretion of the physician. The determination of the administration dosage based on these parameters is well within the capabilities of those skilled in the art. A general administration dosage is 0.001-2000 mg/kg/day, more specifically 0.5-1500 mg/kg/day.

### MODE FOR INVENTION

The examples and experiments will now be described. The following examples and experiments are for illustrative purposes only and not intended to limit the scope of the present disclosure.

### Example 1

### Hot Water Extraction of Green Tea Extract

First flush green tea harvested in Jeju Island of Korea was used. The first flush green tea was isolated and purified by hot water extraction. The detailed hot water extraction procedure is shown in FIG. 1.

Referring to FIG. 1, the flow diagram on the left side is a general tea catechin extraction procedure, the flow diagram at the center is a hot water extraction procedure, and that on the right side is an alcohol extraction procedure.

In this example, the first flush green tea was extracted by hot water extraction, which comprises addition of 5 times the weight of a solvent (water) to first flush green tea leaves, hot water extraction at 50-80° C. for 0.5-12 hours, filtering, vacuum concentration and spray drying.

### Test Example 1

### Analysis of Sample Composition

The composition of the first flush green tea extract prepared in Example 1 was investigated. Specifically, the contents of catechins, amino acids and caffeine were analyzed by the Healthy & Functional Food Research Center. The analysis result is shown in Tables 1, 2 and 3.

TABLE 1

| | Ordinary | First flush | Tea |
|---|---|---|---|
| Content (%) | green tea | green tea | catechin |
| Histidine (His) | 0.98 | 3.97 | — |
| Asparagine (Asn) | 4.16 | 5.10 | 0.58 |
| Serine (Ser) | 0.85 | 1.59 | 0.03 |
| Glutamine (Gln) | 1.61 | 1.37 | — |
| Arginine (Arg) | 0.80 | 3.03 | 8.70 |
| Glycine (Gly) | 0.36 | 0.42 | — |
| Aspartic acid (Asp) | 3.90 | 4.42 | 0.03 |
| Glutamic acid (Glu) | 6.15 | 6.66 | 0.33 |
| Threonine (Thr) | 0.45 | 0.67 | — |
| Alanine (Ala) | 0.64 | 0.61 | — |
| Proline (Pro) | 0.29 | 0.25 | 0.01 |
| Cysteine (Cys) | 0.90 | 1.65 | — |
| Lysine (Lys) | 0.32 | 0.32 | — |
| Tyrosine (Tyr) | 0.39 | 0.41 | — |
| Methionine (Met) | — | — | — |
| Valine (Val) | 0.33 | 0.30 | 0.07 |
| Isoleucine (Ile) | 0.28 | 0.24 | — |
| Leucine (Leu) | 0.23 | 0.27 | 0.08 |
| Phenylalanine (Phe) | 0.39 | 0.41 | 0.06 |
| Tryptophan (Trp) | 0.35 | 0.50 | — |
| Theanine | 1.89 | 2.97 | 0.00 |
| Total amino acid | 4.23 | 6.19 | 0.99 |

TABLE 2

| | Ordinary | First flush | Tea |
|---|---|---|---|
| Content (%) | green tea | green tea | catechin |
| Epigallocatechin | 8.82 | 8.33 | 12.21 |
| (EGC) | | | |
| Epicatechin (EC) | 2.32 | 2.05 | 5.92 |
| Epigallocatechin | 6.39 | 11.36 | 38.47 |
| gallate (EGCG) | | | |
| Epicatechin gallate | 1.68 | 2.43 | 7.22 |
| (ECG) | | | |
| Others | 7.56 | 6.69 | 8.54 |
| Total catechin | 26.77 | 30.86 | 72.38 |

TABLE 3

| Content (%) | Ordinary green tea | First flush green tea | Tea catechin |
|---|---|---|---|
| Caffeine | 2.12 | 2.81 | 0.41 |

The values shown in Tables 1, 2 and 3 are contents in wt % per 1 g of the extract.

As seen from Table 1, the first flush green tea extract had a total amino acid content of 6.19% and a theanine content of 2.97%. In contrast, the ordinary green tea extract had a total amino acid content of 4.23% and a theanine content of 1.89%. The first flush green tea extract had a total catechin content of 30.85% and an epigallocatechin gallate (EGCG) of 11.36%, whereas the ordinary green tea extract had a total catechin content of 26.77% and an EGCG content of 6.39%. Also, the first flush green tea extract had a caffeine content about 25% higher than that of the ordinary green tea extract.

To conclude, the first flush green tea extract had a total amino acid content about 1.5 times higher than that of the ordinary green tea extract. In particular, the theanine content was about 2 times higher. Also, the first flush green tea extract had a total catechin content about 15% than that of the ordinary green tea extract, with the EGCG being about 2 times higher. In addition, the first flush green tea extract had a higher caffeine content than the ordinary green tea extract.

### Test Example 2

### Triglyceride Degrading Ability in Differentiated Adipocytes

[Step 1] Culturing and Differentiation Inducement of Adipocytes

Mouse undifferentiated 3T3-L1 adipocytes (purchased from ATCC) were cultured in Dulbecco's modified Eagle's medium (DMEM; Lonza, 12-604F, USA) containing 10% calf serum (Gibco Co., USA). While replacing the medium every other day, they were cultured in a 37° C. 10% CO₂ incubator to 80% confluency. Then, after culturing the cells for 48 hours in a medium containing 10% fetal bovine serum (Gibco Co., USA), 0.5 mM 3-isobutyl-1-methylxanthine (Sigma Co., USA), 1 μM dexamethasone (Sigma Co., USA) and 167 nM insulin (Sigma Co., USA), followed by replacing the medium with DMEM containing 10% fetal bovine serum and 167 nM insulin, the cells were further cultured for 48 hours. Finally, they were further cultured for 48 hours in a medium only containing 10% fetal bovine serum to obtain differentiated adipocytes.

[Step 2] Treatment to Differentiated Adipocytes

After the inducement of full differentiation, the adipocytes were isolated from the medium, washed with PBS and cultured for 24 hours in a 10% CO₂ incubator in low-glucose DMEM (1000 mg/L D-glucose, without L-glutamine or phenol red; LM001-04, Welgene, Korea) containing 2% free fatty acid bovine serum albumin (Sigma Co., USA). The low-glucose DMEM was treated with tea catechin (70%, PFI Co., Japan) for the positive control group, hot water extract of ordinary green tea (BTC, Korea) for the negative control group, or hot water extract of first flush green tea harvested in Jeju Island (Bioland, Korea) for the test group, at concentrations of 50, 100 and 200 ppm. Then, the cells were cultured at 37° C. in a 10% CO₂ incubator.

[Step 3] Measurement of Fat Degrading Ability after Treatment to Fully Differentiated Adipocytes

The cells cultured in the step 2 were recovered from the medium, seeded on a microplate, 50 μL per each well, and reacted with 50 μL of reaction mixture A of a free fatty acid measurement kit (Roche, Cat #1-383-175, Germany) at 25° C. for 10 minutes. After adding 5 μL of N-ethylmaleimide solution to each well, initial absorbance was measured at 546 nm. Then, 5 μL of reaction mixture B was added to each well and mixed well. After reaction at 25° C. for 15 minutes, final absorbance was measured. Final free acid concentration was determined from the difference of final and initial absorbance with respect to that of blank. The result is shown in FIG. 2.

Referring to FIG. 2, tea catechin showed no fat degrading ability as compared to the control group (media) at a concentration of 50 ppm. In contrast, both the ordinary green tea hot water extract and the first flush green tea hot water extract exhibited fatty acid degrading ability. Also, at 100 ppm, only the first flush green tea hot water extract showed fat degrading ability. At 200 ppm, none of tea catechin, the ordinary green tea and the first flush green tea showed significant effect over the control.

### Test Example 3

### Serum Chemistry Test in Diet-Induced Obesity (DIO) Model

[Step 1] Preparation of Sample

Tea catechin (70%, Pharmafood Inc., Japan) was prepared as control of animal experiment to a concentration of 200 mpk by dissolving in HPLC-grade H₂O (Sigma Co., USA) every day prior to oral administration. The first flush green tea hot water extract (Bioland, Korea) used for the test groups was also prepared by dissolving in HPLC-grade H₂O prior to oral administration at concentrations of 100, 200 and 400 mpk.

[Step 2] Determination of Test Groups and Validation of Body Weight Loss and Fat Degrading Effect

Ten 7-week-old male C57BL/6J mice were prepared per each group. After an accommodation period of 1 week, they were maintained in individual cages under a 12:12-hour light-dark cycle (lights on from 07:00 to 17:00). The groups were: 1) normal diet group (normal feed), 2) high-fat diet group (control), 3) high-fat diet+tea catechin 200 mpk group (tea catechin), 4) high-fat diet+first flush green tea hot water extract 100 mpk group (first flush green tea 100 mpk), 5) high-fat diet+first flush green tea hot water extract 200 mpk group (first flush green tea 200 mpk), and 6) high-fat diet+first flush green tea hot water extract 400 mpk group (first flush green tea 400 mpk). The test substances were given orally at regular hours (10 a.m.) for 8 weeks, once a day. To the 10 mice of the high-fat diet control group, the same volume of water was administered.

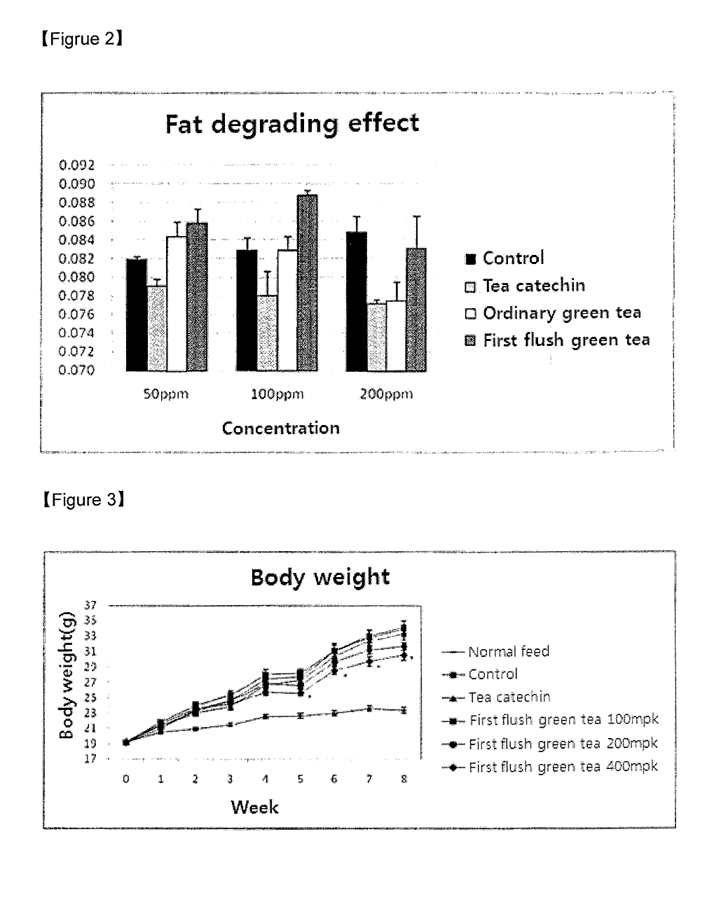

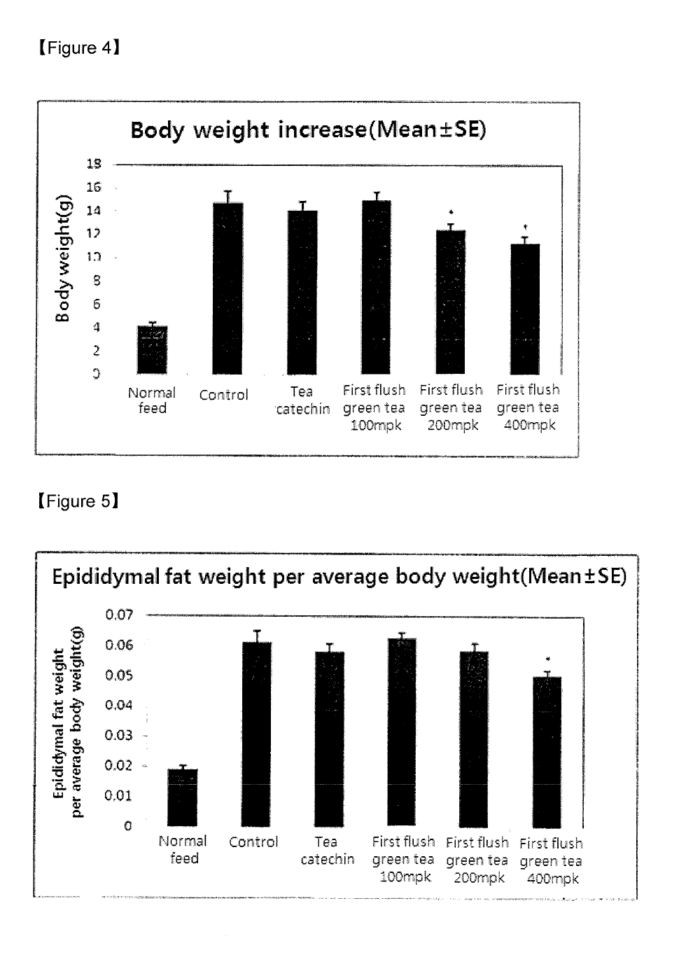

Body weight was measured once a week (at 11 a.m.). The result of measuring the final body weight of the test and control groups on week 8 is shown in FIGS. 3 and 4.

FIG. 3 shows the change in body weight of individual groups, and FIG. 4 shows body weight increase. Referring to FIGS. 3 and 4, the high-fat diet+tea catechin group (tea catechin) showed body weight increase from 19.25±0.69 g to 33.33±2.73 g 8 weeks later, with no statistically significant body weight loss effect as compared to the control group. In contrast, the first flush green tea extract 200 mpk group (first flush green tea 200 mpk) showed body weight increase from 19.12±0.70 g to 31.59±1.46 g 8 weeks later, and the first flush green tea extract 400 mpk group (first flush green tea 400 mpk) showed body weight increase from 19.24±0.68 g to 30.50±2.50 g 8 weeks later. That is to say, the first flush green tea extract according to the present disclosure had statistically significant effect of suppressing body weight increase.

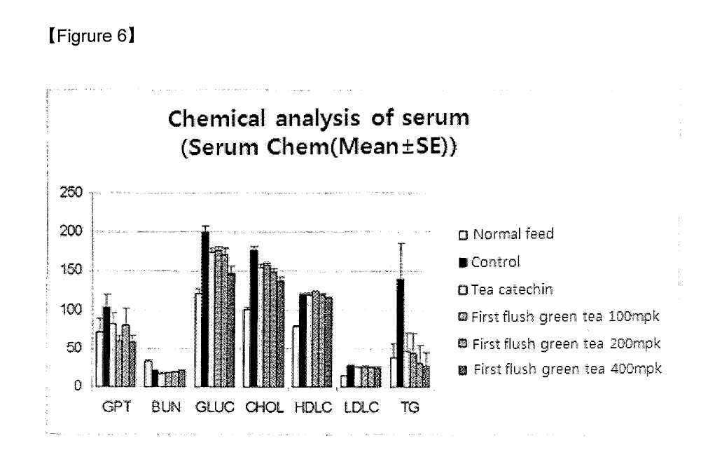

When epididymal fat weight was measured on week 8, the epididymal fat weight of the control group was 2.102±0.170 g, whereas that of the first flush green tea extract 200 mpk group (first flush green tea 200 mpk) was 1.862±0.099 g and that of the first flush green tea extract 400 mpk group (first flush green tea 400 mpk) was 1.543±0.069 g. The measurement result was calculated per average body weight. The result is shown in FIG. 5.

Referring to FIG. 5, the high-fat diet+first flush green tea hot water extract 400 mpk group (first flush green tea 400 mpk) shows statistically significantly lower epididymal fat weight as compared to other groups. Accordingly, it can be seen that the first flush green tea extract according to the present disclosure has fat degrading effect.

[Step 3] Serum Test for Test and Control Groups

Organ toxicity test was carried out for the C57BL/6J mice that had been given tea catechin and the first flush green tea hot water extract at various concentrations for 8 weeks.

In order to investigate the effect on the organs (tissues) of the animal, blood was taken from the animals of the tea catechin- and first flush green tea hot water extract-administered groups and the solvent-administered control group on week 8. Blood glutamate-pyruvate transferase (GPT) and blood urea nitrogen (BUN) levels were measuring using Select E (Vital Scientific NV, the Netherlands). The result is shown in FIG. 6.

In FIG. 6, high-density lipoprotein concentration (HDLC) and low-density lipoprotein concentration (LDLC) are indices showing that obesity was induced normally. All of the control group, the tea catechin group and the first flush green tea groups (first flush green tea 100 mpk, first flush green tea 200 mpk and first flush green tea 400 mpk) showed similar HDLC and LDLC values. Accordingly, it is confirmed that obesity was normally induced in all the high-fat diet groups (control, tea catechin, first flush green tea 100 mpk, first flush green tea 200 mpk and first flush green tea 400 mpk) except for the normal diet group (normal feed). GPT is an indicator of hepatotoxicity and BUN is an indicator of nephrotoxicity.

The test groups (tea catechin, first flush green tea 100 mpk, first flush green tea 200 mpk and first flush green tea 400 mpk) showed no significant difference in GPT and BUN from the control group. Also, when the livers and kidneys were taken out from the animals and subjected to histological observation under an optical microscope following preparation of tissue sections, no special abnormality was observed. Accordingly, the first flush green tea extract according to the present disclosure does not have special toxicity.

Glucose (GLUC) is an indicator of blood sugar. A higher level is often associated with diabetes. Triglyceride (TG) is a blood lipid component causing arteriosclerosis together with cholesterol. Also, cholesterol (CHOL) is a diagnostic indicator of obesity, liver disease and diabetes. As seen from FIG. 6, the first flush green tea groups (first flush green tea 100 mpk, first flush green tea 200 mpk and first flush green tea 400 mpk) showed lower GLUC and CHOL levels as compared to the control group in a concentration-dependent manner. Accordingly, it can be seen that the composition according to the present disclosure is effective for the treatment and prevention of diabetes and obesity.

Also, as seen from FIG. 6, the first flush green tea groups (first flush green tea 100 mpk, first flush green tea 200 mpk and first flush green tea 400 mpk) showed remarkably lower TG level as compared to the control group in a concentration-dependent manner. Accordingly, it can be seen that the composition according to the present disclosure has the effect of reducing CHOL and TG levels and thus is effective for the treatment and prevention of hyperlipidemia, hypertension, arteriosclerosis, angina and myocardial infarction.

The composition comprising the first flush green tea extract according to the present disclosure can be prepared into various formulations as follows, although not being limited thereto.

### Formulation Example 1

### Soft Capsule

Soft capsule was prepared according to the commonly employed method by mixing 100 mg of first flush green tea extract with 50 mg of soybean extract, 180 mg of soybean oil, 50 mg of red ginseng extract, 2 mg of palm oil, 8 mg of hydrogenated palm oil, 4 mg of yellow beeswax and 6 mg lecithin and filling 400 mg of the mixture per each capsule.

### Formulation Example 2

### Tablet

100 mg of first flush green tea extract was mixed with 50 mg of soybean extract, 100 mg of glucose, 50 mg of red ginseng extract, 96 mg of starch and 4 mg magnesium stearate. After adding 40 mg of 30% ethanol, the resulting granules were dried at 60° C. and made into tablets.

### Formulation Example 3

### Granule

100 mg of first flush green tea extract was mixed with 50 mg of soybean extract, 100 mg of glucose, 50 mg of red ginseng extract and 600 mg of starch. After adding 100 mg of 30% ethanol, the resulting granules were dried at 60° C. and filled in a pouch. The final weight was 1 g per pouch.

### Formulation Example 4

### Drink

100 mg of first flush green tea extract was mixed with 50 mg of soybean extract, 10 g of glucose, 50 mg of red ginseng extract, 2 g of citric acid and 187.8 g of purified water and filled in a bottle. The final volume was 200 mL per bottle.

[Formulation Example 5] Health food

| | | |
|---|---|---|
| First flush green tea extract | 1000 | mg |
| Vitamin mixture | | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |
| Mineral mixture | | |
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

The foregoing composition of vitamin and mineral mixtures is an example suitable for health food. The composition may be varied differently. According to the commonly employed method, the described ingredients were mixed to prepare granules for use as health food.

[Formulation Example 6] Health drink

| | | |
|---|---|---|
| First flush green tea extract | 1000 | mg |
| Citric acid | 1000 | mg |
| Oligosaccharide | 100 | g |
| Plum concentrate | 2 | g |
| Taurine | 1 | g |
| Purified water | to make 900 | mL |

According to the commonly employed method, the described ingredients were mixed and heated at 85° C. for about 1 hour with stirring. The resulting solution was filtered, put in a sterilized 2-L container, sealed and sterilized, and kept in a refrigerator for use as health drink.

The foregoing composition is an example suitable for favorite drink. The composition may be varied differently considering age or nationality of consumers, purpose of use, regional or ethnic preferences, or the like.

Those skilled in the art will appreciate that the conceptions and specific embodiments disclosed in the foregoing description may be readily utilized as a basis for modifying or designing other embodiments for carrying out the same purposes of the present disclosure.

### INDUSTRIAL APPLICABILITY

The composition according to the present disclosure comprising a green tea extract as an active ingredient may be widely applicable, for example, in the field of food and medicine.

1-20. (canceled)

21. A method for treating or preventing adult disease of a subject comprising
administering to the subject an effective amount of a first flush green tea extract that treats or prevents adult disease in the subject.

22. The method according to claim 21, wherein the adult disease comprises at least one selected from the group consisting of obesity, diabetes, hyperlipidemia, hypertension, arteriosclerosis, angina and myocardial infarction.

23. The method according to claim 21, wherein the first flush green tea extract is a hot water extract or a C₁-C₅ lower alcohol extract.

24. The method according to claim 23, wherein the C₁-C₅ lower alcohol extract is an ethanol extract.

25. A method for treating or preventing adult disease of a subject comprising
administering to the subject an effective amount of a green tea extract that treats or prevents adult disease in the subject, wherein a total catechin content of the green tea extract is 20-40 wt % based on the total weight of the extract.

26. The method according to claim 25, wherein the adult disease comprises at least one selected from the group consisting of obesity, diabetes, hyperlipidemia, hypertension, arteriosclerosis, angina and myocardial infarction.

27. The method according to claim 25, wherein the green tea extract is a hot water extract or a C₁-C₅ lower alcohol extract.

28. The method according to claim 27, wherein the C₁-C₅ lower alcohol extract is an ethanol extract.

29. The method according to claim 25, wherein the catechin included in the green tea extract comprises epigallocatechin gallate (EGCG), and the content of EGCG is 7-20 wt % based on the total weight of the extract.

30. The method according to claim 29, wherein the green tea extract comprises 2.5-4.5 wt % of caffeine based on the total weight of the extract.

31. The method according to claim 29, wherein a total amino acid content of the green tea extract is 4.5-10 wt % based on the total weight of the extract.

32. The method according to claim 31, wherein the amino acid comprises theanine, and the content of theanine is 2-5 wt % based on the total weight of the extract.
